(19) United States
(12) Patent Application Publication
(10) Pub. No.: US 2025/0327820 A1
(43) Pub. Date: Oct. 23, 2025

(54) DEVICE FOR BLOOD COAGULATION STATUS MONITORING

(71) Applicant: Board of Regents, The University of Texas System, Austin, TX (US)

(72) Inventors: Jing Yong Ye, San Antonio, TX (US); John Forshay, JR., San Antonio, TX (US); Luis Trabucco, San Antonio, TX (US)

(21) Appl. No.: 19/187,856

(22) Filed: Apr. 23, 2025

Related U.S. Application Data
(60) Provisional application No. 63/637,719, filed on Apr. 23, 2024.

(51) Int. Cl. G01N 33/86; G01N 21/41

(52) U.S. Cl. CPC G01N 33/86; G01N 21/41

(57) ABSTRACT

Methods and devices are directed to PT and aPTT testing tailored for home care or point-of-care applications. The approach is based on a unique detection mechanism that measures the changes in the refractive index of a blood sample during its coagulation process in a capillary. The methods and/or associated devices provide patients with the ability to monitor their clotting status conveniently at home.

### RELATED APPLICATIONS

This Application is a US Utility application claiming priority to U.S. Provisional Application 63/637,719 filed on Apr. 23, 2024 which is incorporated herein by reference in its entirety.

### STATEMENT REGARDING FEDERALLY FUNDED RESEARCH

None.

### FIELD

Aspects of the invention are generally directed to the field of medicine and hematology, and particularly the field of blood assessment.

### BACKGROUND

The prothrombin time (PT) test and activated partial thromboplastin time (aPTT) test are indispensable tools in clinical hematology, playing a pivotal role in assessing blood clotting ability and guiding the diagnosis and management of various bleeding disorders, thrombotic conditions, and anticoagulant therapy. Anticoagulant therapy, also known as blood thinning therapy, is a treatment aimed at preventing the formation of blood clots or reducing the risk of existing blood clots growing larger. Blood clots can be dangerous as they can block blood flow to vital organs, leading to serious complications such as stroke, heart attack, or pulmonary embolism. Millions of individuals in the U.S. are prescribed anticoagulant medications (such as warfarin and heparin) to manage conditions such as atrial fibrillation, deep vein thrombosis, pulmonary embolism, and heart valve disorders.

Patients on anticoagulant therapy require careful monitoring of their blood coagulation properties to ensure that the medication is effective, and that the dosage is appropriate. The PT test evaluates the extrinsic and common pathways of the coagulation cascade, primarily assessing the activity of factors I (fibrinogen), II (prothrombin), V, VII, and X. In contrast, the aPTT test primarily evaluates the intrinsic pathway, which involves factors XII, XI, IX, VIII, X, V, and II, as well as prekallikrein and high molecular weight kininogen. Conventionally, these tests are performed in clinical laboratory settings using automated or manual methods involving the addition of specific reagents to plasma samples, followed by the measurement of clotting time using coagulometers. While these methods have proven efficacy, they are often limited by their reliance on specialized equipment and trained personnel, hindering accessibility and timeliness of results, particularly for individuals requiring frequent monitoring or residing in remote areas. As a result, there is a pressing need for innovative approaches that enable convenient, reliable, and user-friendly PT and aPTT testing for home care or point-of-care settings.

### SUMMARY

Embodiments of the current application address the limitations imposed by reliance on specialized equipment and trained personnel, particularly for individuals requiring frequent monitoring or residing in remote areas. The solution to this problem includes a novel approach for assessing blood properties, e.g., PT and aPTT testing, tailored for home care or point-of-care applications. This approach is based on a unique detection mechanism that measures the changes in the refraction angle of a probe beam refracted from a blood sample in a capillary during its coagulation process. The term “capillary” or “capillary tube” refers to a tube of small internal diameter that loads and holds liquid by capillary action. The methods and devices described herein can enhance patient engagement, improve treatment adherence, and facilitate timely intervention, ultimately contributing to better clinical outcomes for individuals managing coagulation disorders by providing patients with the ability to monitor their clotting status conveniently at home.

Certain embodiments are directed to an approach for PT and aPTT testing tailored for home care or point-of-care applications. The approach is based on a unique detection mechanism that measures the changes in the refractive index of a blood sample during its coagulation process in a capillary. The refractive index of blood is a measure of how much light bends when passing through it, influenced by its composition, including red blood cells, plasma, and other constituents. It typically ranges from 1.35 to 1.41 at visible wavelengths (around 400-700 nm), with an average value of approximately 1.38 for whole blood at 589 nm (sodium D-line). In certain aspects the refractive index is indicative of various disease states, including but not limited to coagulopathies. Methods and/or associated devices described herein provide patients with the ability to monitor their clotting status conveniently at home.

Certain embodiments are directed to blood coagulation monitoring devices. The devices include a divergent probe beam source; a sample holder, the sample holder configured to position a capillary containing a sample offset from the center of the divergent probe beam; a sensor configured to detect a portion of probe beam; the probe beam source and capillary are positioned such that a portion of the divergent probe beam passes through the capillary containing the sample and converges onto the sensor. A divergent probe beam is a beam where the beam diameter or radius increases as the distance from the source increases. The divergent probe beam source can be a diode laser or a lens-coupled laser source. The sensor can be an imaging chip or other sensor configured to detect probe beam deflection. In certain aspects the device can further include a control unit operably connected to the sensor. The control unit can be programmed to carryout the various tasks and provide one or more of data input/output module, storage module, a user interface and/or communication module.

Other embodiments are directed to a blood collection capillary comprising a collection segment coated with calcium chelator or other coagulation inhibitor and a detection segment coated with a coagulation activator, wherein the capillary is configured to receive a sample in the collection segment and during operation the sample is transferred to the detection segment. The calcium chelator or coagulation inhibitor can be citrate or oxalate. The coagulation activator can be CaCl₂, calcium gluconate, silica, celite, kaolin, or ellagic acid. In certain aspects the inner diameter of the capillary can be between 0.1, 0.2, 0.3, 0.4, 0.5, 0.6, 0.7, 0.8, 0.9, 1.1, 1.2, 1.3, 1.4, 1.5, 1.6, 1.7, 1.8, 1.9, 2.1, 2.2, 2.3, 2.4, 2.5, 2.6, 2.7, 2.8, 2.9, to 3 mm, including all values and ranges there between. In other aspects the volume of the detecting segment can be between 1, 2, 3, 4, 5, 6, 7, 8, 9, to 10 μL.

Other embodiments are directed to methods for monitoring blood coagulation. The methods including one or more steps from (i) exposing a capillary containing a blood sample to a diverging probe beam that is configured to pass through the blood sample, the capillary converging the diverging probe beam, and the converged probe beam impinging on a sensor configured to detect changes in refraction of a portion of the diverging probe beam; (ii) detecting changes in refractive index (e.g., rate of coagulation) of the sample by detecting a change in refraction angle of the probe beam as determined by analyzing data collected by the sensor; (iii) assessing blood coagulation processes in the sample based on the change in refraction angle of the diverging probe beam as it passes through the sample to determine the change in refractive index of the blood sample. In certain aspects the sensor can be an imaging chip. The sensor can be configured to collect data continuously over a period of time. The sample volume analyzed can be between 1 to 10 μL.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The term “Coagulation” refers to the biological process by which blood transforms from a liquid to a gel-like state, forming a clot to prevent excessive bleeding and initiate wound healing. It involves a complex cascade of events triggered by vascular injury, where platelets aggregate at the site of damage and a series of enzymatic reactions among clotting factors-primarily proteins in the blood plasma-culminate in the conversion of fibrinogen into fibrin. Fibrin forms a mesh that stabilizes the platelet plug, creating a durable clot. This process is tightly regulated by procoagulant and anticoagulant mechanisms to ensure clots form only where needed and are later dissolved during tissue repair. Disruptions in coagulation can lead to conditions like hemophilia (insufficient clotting) or thrombosis (excessive clotting).

The term “monitoring device” refers to an instrument or system designed to continuously or periodically measure, record, and analyze specific parameters or conditions of a subject, sample, or environment to provide real-time or retrospective data. In medical settings, such devices may monitor physiological parameters (e.g., heart rate, blood glucose, or oxygen saturation) or, in laboratory contexts, properties of a sample (e.g., blood coagulation, chemical composition). Typically, a monitoring device integrates sensors, data processing units, and output interfaces (e.g., displays or data logs) to enable accurate tracking and interpretation.

The term “divergent probe beam” refers to a beam of light, typically emitted from a source such as a laser or LED, that spreads out or diverges as it propagates through a medium, increasing in cross-sectional area over distance. Unlike a collimated beam, which maintains a constant diameter, a divergent probe beam emanates from a point source or an optical system designed to produce a conical or fan-shaped light pattern, with the beam's divergence angle determined by the source's properties or the optics used. In scientific and engineering applications, such as optical sensing, spectroscopy, or medical diagnostics, a divergent probe beam is often employed to illuminate a sample, allowing for the probing of a larger area or volume to collect scattered, reflected, or transmitted light for analysis. The divergence characteristics can be used for optimizing signal detection, resolution, and the interaction of light with the target material or biological sample.

The term “Sample holder” refers to a device or component designed to securely contain, position, or stabilize a specimen, such as a blood sample, tissue, or other material, during analysis, imaging, or testing in medical, scientific, or diagnostic applications. Typically constructed from materials like plastic, metal, or glass, a sample holder ensures precise alignment of the sample within an analytical system, such as a microscope, spectrometer, or coagulation analyzer, to facilitate accurate measurements or observations.

The term “Capillary” refers to a narrow tube or channel, typically made of materials like glass, plastic, or metal, designed to mimic the dimensions and fluid dynamics of biological capillaries or to facilitate the controlled movement of small fluid volumes, such as blood or other liquids, via capillary action or external forces. Unlike biological capillaries, mechanical capillaries are artificial structures used in devices like microfluidic systems, diagnostic cartridges, or analytical instruments, where their small internal diameter (often in the micrometer to millimeter range) enables precise fluid handling, sample collection, or separation of components (e.g., plasma from whole blood). A mechanical capillary may be defined by its geometry, surface (internal or external) properties (e.g., hydrophilicity), or integration with sensors or activators, distinguishing it for applications in point-of-care diagnostics, optical analysis, or fluidic circuits.

The term “diode laser” refers to a compact, efficient device that generates coherent light through the process of stimulated emission within a semiconductor material, typically gallium arsenide or similar compounds. It operates by passing an electric current through a p-n junction, where electrons and holes recombine, releasing energy as photons. The emitted light is amplified within a resonant cavity formed by the cleaved facets of the semiconductor or external mirrors, producing a highly focused, monochromatic beam. Diode lasers are characterized by their small size, low power consumption, and ability to emit light across a range of wavelengths, from ultraviolet to infrared, depending on the material composition. Widely used in applications such as telecommunications, barcode scanners, medical treatments, and optical sensing, diode lasers are valued for their reliability, tunability, and integration into electronic systems.

The term “imaging clip” refers to a specialized device or component designed to secure, position, or hold a sample, such as a blood sample in a capillary or a tissue specimen, for imaging or analytical purposes. Typically used in conjunction with optical, spectroscopic, or microscopic systems, an imaging clip ensures precise alignment and stability of the sample within the imaging field, minimizing movement or distortion during analysis.

The term “Coagulation activator” refers to a substance or agent used to initiate or accelerate the blood clotting process, typically in vitro, for diagnostic or research purposes in medical and laboratory settings. These activators trigger the coagulation cascade by promoting platelet aggregation or activating clotting factors, leading to the conversion of fibrinogen into fibrin and the formation of a stable clot. Common coagulation activators include substances like kaolin, celite, glass particles, or tissue factor (thromboplastin), which mimic natural triggers of coagulation such as vascular injury. In clinical applications, such as in coagulation assays (e.g., activated partial thromboplastin time or prothrombin time tests), these activators are added to a blood sample to standardize and measure the clotting time, aiding in the diagnosis of hemostatic disorders or monitoring anticoagulant therapies.

The term “data” refers to a collection of quantitative or qualitative information generated, recorded, or processed during the observation, measurement, or analysis of a sample, subject, or system. This information may include numerical values (e.g., clotting times, optical intensities), images (e.g., from an imaging clip), or other measurable outputs (e.g., spectral readings from a diode laser) obtained through devices including monitoring systems or sample holders.

The term “blood sample” refers to a quantity of blood, typically obtained from a human or animal subject through venipuncture, fingerstick, or other collection methods, intended for diagnostic, analytical, or research purposes. The blood sample may comprise whole blood, plasma, serum, or specific cellular components (e.g., red blood cells, white blood cells, or platelets). Such samples are often processed, stabilized, or stored under controlled conditions to preserve their integrity for testing, which may include biochemical, hematological, or molecular analyses.

The term “refractive index” refers to a dimensionless physical property of a material that quantifies how light propagates through it compared to a vacuum. Defined as the ratio of the speed of light in a vacuum to the speed of light in the material (n=c/v), it indicates the degree to which light bends or refracts when entering or passing through the medium. The refractive index depends on the material's composition, density, and the wavelength of light, typically ranging from 1 (for a vacuum) to higher values for denser media like glass (1.5) or water (1.33). In applications such as optics, medical imaging, or material science, the refractive index is critical for designing lenses, analyzing biological samples, or characterizing substances, as it influences phenomena like reflection, refraction, and dispersion.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Schematic drawing of one example of a system for monitoring the changes in the refractive index of the analyte sample contained in a glass capillary. The dashed line indicates the shift of the beam focusing due to the change in the refractive index of the analyte sample in the capillary.

FIG. 2A-2C. Schematic drawing of two examples of approaches for sample handling. (A) The blood sample is first collected into the capillary first section coated with citrate, which prevents blood coagulation. The blood sample is then transferred to the detection region by pulling a plunger controlled by a motor. (B) The second approach for handling the blood sample is to use an air chamber connected with a Peltier module. When the chamber is cooled down, the negative pressure pulls the blood sample into the detection zone coated with Tissue Factor and CaCl₂. (C) The third approach for handling the blood sample is to connect the capillary containing the collected blood sample with a pump, which can transfer the blood sample to the detection region. The first region initially containing the blood sample may or may not be coated with citrate. Accordingly, the detection zone may be coated with Tissue Factor and CaCl₂ or coated with only Tissue Factor.

FIG. 3A-3B. (A) Top view schematic of the system for coagulation measurements, including a syringe, PDMS holder, a diverging probe laser beam, a neutral density (ND) filter, and an imaging chip. (B) Side view schematic of the system for coagulation measurements showing the vertically offset of the glass capillary tube holding an analyte sample with a representative ray tracing from Zemax simulations of the probe laser beam passing through the sample inside of the capillary.

FIG. 4A-4N. Illustration of the shifts of focused laser lines (A-F) on the experimentally obtained images between water and 10% aqueous ethylene glycol (EG) solution. (G-L) The shifts of the focused laser lines between water and 10% EG solution on the images obtained from Zemax POP simulations. (M) Effects of increasing offset of the capillary tube relative to the laser. Both the experimental (circle) and Zemax simulation (square) results show a linear increase of the relative line shift with increasing the capillary offset. (N) Experimentally obtained relative shift of the focused laser line positions for varying percentages of EG-water mixtures relative to water. The refractive index (RI) of those samples at 650 nm were obtained from calculation based on their relative position to water and acetonitrile, which have known RIs at 650 nm.

FIG. 5. Illustrates an example of continuous measurements of coagulations of high (632 mg/dL) fibrinogen control (circle) and low (93 mg/dL) fibrinogen control (square) plasma initiated with thromboplastin-DS. (Inset) Average t₅₀ for high (left) and low (right) fibrinogen controls (p-value=0.0025).

### DESCRIPTION

The following discussion is directed to various embodiments of the invention. The term “invention” is not intended to refer to any particular embodiment or embodiments or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment and not intended to imply that the scope of the disclosure, including the claims, is limited to that embodiment.

Monitoring coagulation time is a critical component of anticoagulant therapy for patients with conditions such as deep vein thrombosis (DVT), pulmonary embolism (PE), atrial fibrillation (AF), and mechanical heart valve replacements. Warfarin, one of the most widely prescribed oral anticoagulants globally, requires careful dose management to maintain efficacy while minimizing the risk of bleeding or thromboembolic events. Prothrombin time (PT) and its standardized counterpart, the international normalized ratio (INR), are routinely used to assess the extrinsic pathway while the activated partial thromboplastin time (aPTT) test is used to evaluate the intrinsic pathway of coagulation [1]. In outpatient care, the PT test is more commonly used, as warfarin, which affects the extrinsic pathway, is the most commonly prescribed oral anticoagulant for long-term anticoagulation management worldwide [2]. Since PT values can vary between laboratories and methodologies, the INR enables standardized interpretation, facilitating more consistent and effective therapy management.

In the coagulation cascade, prothrombin plays a pivotal role as it is enzymatically converted into thrombin, which subsequently catalyzes the transformation of fibrinogen into active fibrin [3]. This cascade is triggered by factor III (thromboplastin), which initiates a series of proteolytic reactions, ultimately culminating in clot formation [3, 4]. Thrombin cleaves circulating fibrinogen into fibrin monomers, which polymerize and crosslink to form a stable fibrin clot which traps platelets and red blood cells [5, 6]. Warfarin inhibits vitamin K epoxide reductase, an enzyme essential for activating vitamin K-dependent coagulation factors II (prothrombin), VII (proconvertin), IX (Chrismats factor), and X (Stuart factor), thereby extending the time required for clot formation [7, 8]. For healthy individuals, PT values typically range from 9 to 13 seconds, and INR values fall between 0.8 and 1.2 [7, 9]. In anticoagulated patients, INR targets generally range from 2.0 to 3.0, and up to 3.5-4.0 for those with prosthetic heart valves [10].

PT/INR testing has traditionally been conducted in hospitals or outpatient laboratories, using 2.8 mL of whole blood [11]. On-site testing may yield results within hours, while off-site testing may require several days [12]. These delays reduce dosage accuracy and adherence, which can compromise patient outcomes [13]. The problem is particularly acute among elderly and mobility-impaired individuals [14]. In recent years, there remains a demand for more convenient medical diagnostics and treatments. As a result, the need for timely, accessible, and low-volume coagulation monitoring for long-term anticoagulant therapy has increased [15, 16].

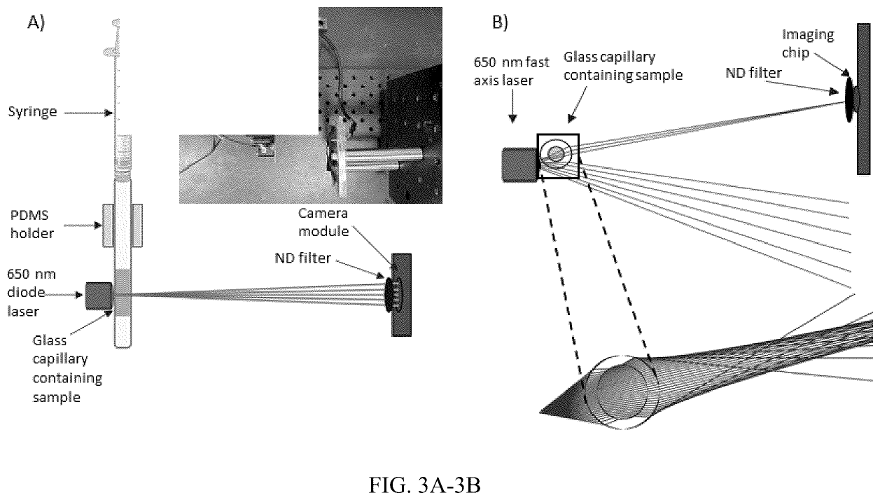

Herein is a novel optical method for quantifying PT/INR based on changes in the refractive index (RI) of a plasma sample undergoing coagulation inside a thromboplastin-coated glass capillary tube or similar device/system. Unlike traditional optical scattering or transmittance techniques, which are highly susceptible to sample clarity and particulate interference [17], this system captures the RI-induced image displacement of a probe laser as the clotting process alters the optical properties of the sample. By optimizing the capillary's vertical offset relative to the laser beam, the system maximizes sensitivity to minute RI changes, offering robust and precise detection of coagulation dynamics (FIG. 3A-3B). This approach offers several advantages over conventional point-of-care (POC) platforms, such as those based on quartz crystal microbalance resonators and electrochemical methods. Quartz sensors are highly sensitive to environmental conditions like humidity and temperature [18], while electrochemical techniques require exact electrode positioning and are subject to signal drift [19]. In contrast, the RI-based method described herein is resilient to these variables and requires no contact with the blood sample beyond containment in the capillary, reducing risk of contamination or operator error.

An additional diagnostic strength of the system is its minimal sample requirement. While current at-home PT/INR testing devices, including the Coag-Sense (CoaguSense Inc.) and CoaguChek XS (Roche Diagnostics), require 8-10 μL of whole blood, the method(s) described herein achieves reliable detection using just 4 μL of plasma. This volume reduction is especially important given the variability in fingerstick blood yields, which depend on patient physiology, lancet size, and technique [20, 21]. The ability to achieve accurate measurements with lower sample volumes positions this system as a significant advancement in patient-centric coagulation monitoring.

Taken together, compositions, devices, systems, and/or methods described herein introduce a sensitive, low-volume, and environmentally robust optical sensing method for PT/INR quantification-addressing key limitations of existing technologies while offering a promising platform for widespread deployment in home and POC settings.

### I. BLOOD COAGULATION MONITORING

Certain embodiments describe a unique mechanism for PT testing using refractive index (RI) measurements of an analyte sample in a thromboplastin-coated glass capillary. The system detects coagulation by monitoring RI-induced image shifts of a probe laser beam focused by the glass capillary tube functioning as a cylindrical lens. The capillary is vertically offset from the principal axis of the diverging laser beam, in which the degree of offset is optimized to enhance the image shift in response to variations in the sample's RI. This design offers several advantages, including minimal sample volume (4 μL), high sensitivity, real-time monitoring, and the potential for rapid point-of-care or at-home PT tests.

Thromboplastin, also known as tissue factor, is a protein found in tissues, particularly within blood vessel walls. When blood vessels are injured, thromboplastin is exposed to blood and initiates the coagulation cascade. It serves as a catalyst for the conversion of prothrombin to thrombin, which is a crucial step in forming a blood clot. Fibrinogen, on the other hand, is a soluble plasma protein synthesized by the liver. During the coagulation process, thrombin acts on fibrinogen, converting it into insoluble fibrin strands. These fibrin strands form a mesh-like network that traps blood cells, platelets, and plasma to form a stable blood clot. During the clotting process, the refractive index of the blood increases with time. Methods and associated apparatus and systems described herein monitor the clotting process using a capillary filled with blood as a focusing cylindrical lens to focus a probe beam onto an imaging chip and measure the change in the refraction angle of the probe beam during the blood coagulation.

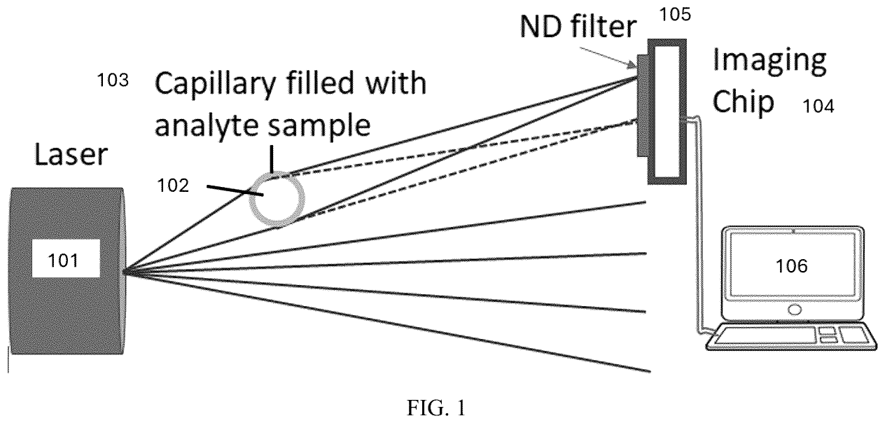

One example of a system for monitoring blood coagulation is illustrated in FIG. 1. A laser or light source 101 (e.g., a diode laser) emits a diverging beam to serve as a probe for the sample 102. A capillary containing the analyte sample 103 is placed within the laser beam but offset from the center of the beam. The distance between the capillary and the laser is meticulously adjusted to allow the diverging laser beam to converge at a certain distance (e.g. 6 cm) away from the capillary after the beam traverses through the capillary. An imaging chip 104 or similar component is placed at the focus of the beam to monitor the position of the beam. A neutral density (ND) filter 105 is placed in front of the imaging chip 104 to control the appropriate amount of light exposure. Images are continuously captured and processed by a computer 106 or other control device to record the shift of the focused beam due to the changes in the refractive index of the sample within the capillary 103.

In this system, the capillary 103 serves multiple functions: (1) Holding the analyte sample 102. Due to the small inner diameter of the capillary selected, typically around 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, to 3 mm (including all values and ranges there between). In certain aspects the capillary inner diameter can be between 0.5 and 1.5 mm. A minimal amount of sample equivalent to a small drop of blood is required, e.g., 1, 2, 3, 4, 5, 6, 7, 8, 9, to 10 μL, including all values and ranges there between. In certain aspects the sample volume is 2 μL or less (0.1 to 2 μL). This stands in contrast to the approximately 1 mL of blood typically needed for PT or aPTT tests conducted in clinical laboratories. One benefit of the methods, apparatus, capillary, or system described herein is minimization of the required amount of sample (e.g., less than 2 to 5 μL) so that subjects or patients can obtain the blood sample at home by simply lancing their finger. (2) Acting as a cylindrical lens to focus the probe beam. To precisely measure the change in the deflection angle of the probe beam once it passes through the analyte sample, the beam needs to be focused down and detected, e.g., imaged with an imaging chip or the like. Utilizing the inherent curvature of the capillary, when combined with the filled sample, serves as a cylindrical lens, effectively focusing the probe beam onto a sensor, e.g., complementary metal-oxide semiconductor (CMOS) imaging chip, without the need for additional lenses. This streamlined setup simplifies the configuration and enables the construction of a compact system. (3) Enhancing the detection sensitivity by magnifying the changes in the probe beam position. Since the capillary is placed off the center of the probe beam, the focused beam position has a vertical shift when the refractive index of the analyte sample changes. In addition, as the probe beam is focused down to a thin line by the capillary as a cylindrical lens, a higher accuracy of determining the beam position can be achieved with the imaging chip compared to an unfocused beam.

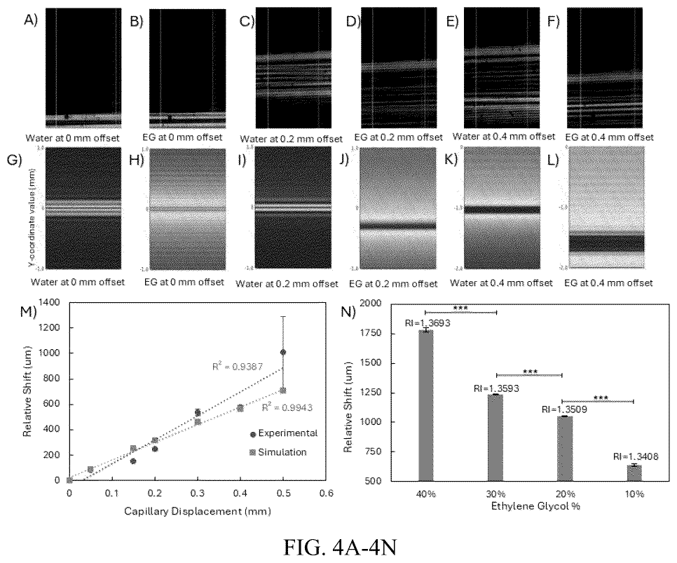

An extremely high sensitivity of quantifying the refractive index of analyte samples with this system has been demonstrated. The change of refractive index has been obtained in a model system by mixing ethylene glycol with water. The refractive index of water at 650 nm is 1.3314, while that is 1.4291 for ethylene glycol. The results show that the probe beam position sensitively shifts with changing the percentage of ethylene glycol in water (FIG. 4N), i.e., with changing the effective refractive index.

As the system only requires a minimum amount (e.g., 2 μL) of blood sample, a common procedure for collecting a drop of blood can be performed at home by a subject or in a clinical setting for the PT or aPTT test. Typically, the fingertip or an alternate site such as the forearm or palm is a suitable site for blood collection. A lancet can be loaded into a lancet device and the depth setting on the lancet device can be adjusted to a relatively shallow depth taking advantage of the small amount of sample required, which may help minimize discomfort for the patient. The lancet device can be placed against the cleaned skin site, and upon releasing the button, it swiftly punctures the skin. Subsequently, a capillary can be held against the drop of blood to collect the sample through capillary effects.

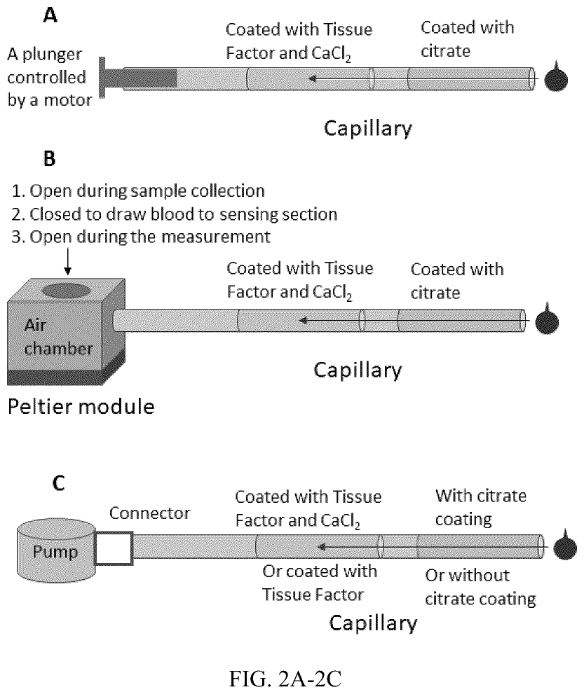

The first section or segment of the capillary is coated with a calcium chelator, such as citrate, to inhibit blood coagulation. After the blood sample is collected to the first (collection) section or segment, the capillary is inserted into a designated device. Two methods can be employed to transport the blood to the second (detection) section or segment, where the capillary is coated with calcium chloride and tissue factor or other coagulation initiator to trigger coagulation for PT or aPTT tests (FIG. 2). The first method is to utilize a plunger, which is controlled by a motor, to pull the blood sample from the first section further into the section coated with coagulation initiator, e.g., tissue factor and CaCl₂. The detection is then started to measure the change in the probe beam position with time. The second approach is to have a small chamber connected with the capillary. The chamber is open when the capillary is used to collect the blood sample, while the chamber is closed when it is placed on a Peltier module inside the device before the detection of the coagulation process. The Peltier module is utilized to decrease the temperature within the chamber, generating a negative pressure that draws the blood sample into the capillary section or segment coated with the coagulation initiator, e.g., tissue factor and CaCl₂. Employing the Peltier module for sample handling eliminates the need of any moving components (such as a motor and a plunger), thereby enhancing the device's robustness and reducing complexity. As the Peltier module is a low-cost component, this approach can also reduce the cost of the device. Alternatively, the capillary containing the collected blood sample may be connected with a pump, which can transfer the blood sample to the detection region. The first region initially containing the blood sample may or may not be coated with citrate. Accordingly, the detection zone may be coated with Tissue Factor and CaCl₂ when the first region is coated with citrate, while the detection zone may be coated with Tissue Factor when the first region is not coated with citrate.

The probe beam refracted from the capillary is imaged with an imaging chip. The shift of the probe beam position with time is recorded by a computer connected to a sensor, e.g., an imaging chip, or by a Raspberry Pi (a small single-board computer). Two different methods may be utilized to determine the coagulation time of the blood sample. One is to set a threshold for the number of pixel changes of the probe beam relative to its original position before coagulation. The time duration for the probe beam position to reach the threshold is defined as the coagulation time. The second method is to fit the entire data points of the probe beam positions as a function of time. The time constant from the fitting represents the coagulation time.

The system described herein demonstrates a sensitive response to the change in the refractive index of analyte samples. With a required sample volume as small as 2 μL, smaller than a single drop of blood, the system presents a novel detection mechanism for PT and aPTT tests based on monitoring the change in the refraction angle of a probe beam, potentially enabling the quantification of blood coagulation in a patient's home or a doctor's office.

### II. MONITORING AND INDICATING DISEASES

In certain aspects the device described herein can be used to monitor or indicate various diseases. Diseases of interest include those which may disrupt blood coagulation and/or diseases which do not indirectly or directly impact blood coagulation. In some cases a medication is administered to treat a disease that can impact blood coagulation.

Blood coagulation, a critical physiological process, becomes dysregulated in various diseases, leading to either excessive bleeding or inappropriate clot formation. Normally, coagulation involves a cascade of enzymatic reactions culminating in fibrin formation, which stabilizes platelet plugs to stop bleeding. However, in diseases like hemophilia, deficiencies in clotting factors (e.g., factor VIII or IX) impair this cascade, resulting in prolonged bleeding even from minor injuries. Hemophilia patients often experience spontaneous joint and muscle bleeds, requiring factor replacement therapies to restore hemostatic balance. Conversely, in conditions like disseminated intravascular coagulation (DIC), widespread activation of the coagulation system due to sepsis, trauma, or malignancy consumes clotting factors and platelets, paradoxically causing both thrombosis and bleeding. DIC reflects a systemic imbalance, with microthrombi clogging small vessels while severe depletion of coagulation components leads to hemorrhage.

Liver disease significantly impacts coagulation due to the liver's role in synthesizing clotting factors and anticoagulants. In cirrhosis or acute liver failure, reduced production of factors II, V, VII, IX, and X, alongside thrombocytopenia from splenic sequestration, predisposes patients to bleeding, particularly in the gastrointestinal tract. Yet, these patients can also develop thrombosis due to decreased anticoagulant proteins (e.g., protein C and S) and elevated von Willebrand factor. This dual risk complicates treatment, as standard anticoagulation or procoagulant therapies may exacerbate one issue while addressing the other. Advanced diagnostics, like thromboelastography, are increasingly used to assess real-time coagulation status in such complex diseases, guiding personalized interventions.

Anemia, characterized by low red blood cell count or hemoglobin, indirectly influences blood coagulation by altering blood flow dynamics and compensatory mechanisms. In conditions like iron-deficiency anemia or sickle cell anemia, reduced oxygen-carrying capacity can stress vascular endothelium, promoting inflammation and potentially increasing procoagulant activity. Sickle cell disease, for example, is notably prothrombotic due to sickled red cells causing vascular occlusion, activating platelets, and elevating tissue factor expression. Chronic anemia may also lead to compensatory increases in von Willebrand factor, enhancing platelet adhesion and clot formation. Conversely, severe anemia can impair coagulation by reducing blood viscosity, which affects shear stress needed for platelet activation. In bone marrow disorders like aplastic anemia, thrombocytopenia often accompanies anemia, increasing bleeding risk due to insufficient platelet-driven clot formation. Treatment focuses on addressing the underlying anemia (e.g., iron supplementation, transfusions) while carefully monitoring coagulation status, particularly in patients requiring invasive procedures or those with concurrent thrombotic risks.

Cancer significantly disrupts normal blood coagulation, often inducing a hypercoagulable state that increases the risk of thrombosis. This is driven by tumor cells expressing procoagulant factors, such as tissue factor, which initiates the clotting cascade, and releasing microparticles that promote fibrin formation. For instance, pancreatic, lung, and brain cancers are particularly associated with venous thromboembolism (VTE), including deep vein thrombosis and pulmonary embolism. Inflammatory cytokines like interleukin-6, elevated in cancer, further enhance coagulation by upregulating clotting factors and reducing anticoagulant proteins like protein C. Additionally, treatments like chemotherapy can damage endothelial cells, exacerbating thrombotic risk. However, some cancers, particularly leukemias, can cause coagulopathies like disseminated intravascular coagulation (DIC), where widespread clotting consumes platelets and clotting factors, leading to severe bleeding. Managing coagulation in cancer patients is complex, often requiring low-molecular-weight heparin or direct oral anticoagulants, balanced against bleeding risks, especially in those with thrombocytopenia or metastatic disease.

In certain embodiments the device described herein is used to indicate various diseases, such as those described above. This includes the use of the device to measure the refractive index of the blood. Certain diseases, such as anemia and hypoproteinemia, may lower blood refractive index, while certain diseases, such as polycythemia vera, hyperproteinemia and hyperlipidemia, may increase blood refractive index.

### III. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention and thus can be considered to constitute preferred modes for its practice. However, those of skill in the art should, in view of the present disclosure, appreciate that many changes can be made in the specific embodiments which are disclosed and still obtain a like or similar result without departing from the spirit and scope of the invention.

### A. Materials and Methods

Materials. This optical system is centered around three main components: a 650-nm semiconductor laser, a glass capillary tube (OD 1.6 mm, ID 1.1 mm), and a camera module (Arducam 8MP 1080P USB) for Raspberry Pi (3264 pixels×2448 pixels). A Polydimethylsiloxane (PDMS) holder was molded to fit the OD of the capillary tube by curing the solution while the tube was inside, limiting vertical displacement during experimental procedures. For initial experimental collection, ethylene glycol concentrations of 10%, 20%, 30%, and 40% were loaded into the capillary tube to determine the sensitivity of the system to changes in RI. Plasma coagulation was subsequently measured through initiation by a thromboplastin-DS coating inside the capillary tube. Thromboplastin-DS was obtained from Thermo-Fisher (Product No. 10-0354) and reconstituted to a concentration of 56 μg/mL with deionized water. Control plasmas containing low (Product No. 10-0604) and high (Product No. 10-0607) fibrinogen were obtained from Thermo-Fisher, which are prepared from human plasma collected with a sodium citrate anticoagulant (4% w/v). To prepare the controls for use, the plasma tablet was reconstituted with 1.0 mL of distilled water, swirled gently, and stood undisturbed for 15 minutes at room temperature. Once reconstituted, the High and Low Fibrinogen Controls were stable for up to 8 and 16 hours when stored at 2-8° C. in a capped vial, respectively. PDMS was obtained from Sylgard.

Optical System Setup. The optical system includes a 1-mW, 650-nm semiconductor laser as the light source, with no collimating or focusing lens. The laser was fixed to a micrometer-adjustable stage, allowing minute changes in the height position of the laser relative to the capillary tube (FIG. 3). This vertical offset is a critical parameter to optimize the detection sensitivity as shown in the Results section. An increase in the offset results in an increase in the laser light's angle of refraction, enhancing the system's sensitivity to changes in sample RI. The center of the camera module was fixed 14.13 mm above the principal axis of the laser and 69.2 mm away from the capillary tube to effectively capture the resulting image from the sample contained in the glass capillary tube. As the capillary tube serves as a cylindrical lens, the camera was used without any additional lens. A neutral density filter (OD 1) was placed in front of the camera to mitigate ambient light noise. Lastly, a PDMS holder, which was cured to the outer diameter of the capillaries (1.6 mm), was attached in a position that placed the capillary tube 1.05 mm away from the laser source. This holder was set up in line to a syringe which allowed for control of pressure changes to move samples through the capillary tube (FIG. 3A). To optimize image quality, the focal length was determined experimentally through a deionized water sample loaded inside of the capillary tube.

Zemax Simulation Setup. For numerical simulations of the optical configuration, an initial schematic including a fast-axis diverging laser beam and a capillary-sample assembly was created (FIG. 3A) and then replicated in Ansys Zemax OpticStudio, an optical design software, to simulate the image formation of the probe laser beam passing through the sample. Sequential ray tracing was used to approximate and visually represent the beam refraction by the sample contained in the capillary tube (FIG. 3B). The capillary-sample assembly effectively acts as a cylindrical lens to focus the diverging beam to a line on an imaging chip. For a 2D representation, each axis of the laser was modeled as a Gaussian beam with a wavelength of 650 nm and a 2 mm float-by-stop aperture. The capillary tube, represented as a toroidal BK-7 glass component containing a sample with variable RI, was optimized to be 1.05 mm away from the laser source, and then vertically decentered incrementally (0-0.5 mm) from the principal axis to investigate the detection sensitivity as a function of the vertical offset. The imaging plane was positioned 69.2 mm away from the capillary tube, with aperture settings matching the actual imaging chip dimensions. The capillary tube was modeled with outer and inner diameters of 1.6 mm and 1.1 mm, respectively. Sample material RI values at 650 nm were assigned based on experimentally obtained RI values of ethylene glycol-water mixtures with varying ratios.

To assess the sensitivity of the probe beam's focal shift in response to changes in the RI of a test sample, different offsets of the capillary tube relative to the probe beam's principal axis were modeled in Zemax. Sequential ray tracing alone, however, does not account for realistic variability such as total internal reflection (TIR), non-symmetrical axis divergencies, and vignette rays that do not interact with the capillary tube at all. To better model this device, a Physical Optics Propagation (POP) analysis was conducted to evaluate irradiance distribution on the imaging plane. In this analysis, the beam was defined as a Gaussian beam with divergence angles of 5.13° and 21.1° (determined experimentally) on the X (slow axis) and Y (fast axis) axes, respectively. A 2048×4096-pixel (x, y) sampling size was used to generate the irradiance map on the imaging chip.

Plasma Coagulation. Freeze-dried plasma tablets and thromboplastin-DS were purchased from Thermo-Fisher. Glass capillaries were loaded with 2 μL of an aqueous thromboplastin-DS solution (56 μg/mL) and left to dry (2 days at 2° C.) for coating the interior wall. Coagulation results were obtained using the same batch of capillaries. This ensured that the plasma coagulation was initiated by thromboplastin-DS which had dried in the same manner as well as under the same conditions. For each test, 4 μL of plasma solution was pulled into the capillary tube via the capillary effect in front of the laser where coagulation was induced. Data was acquired for approximately 120 sec—well after the image shifting stopped.

A custom MATLAB program was used to obtain coagulation data in real time as well as initial experimental data covering the RIs of different ethylene glycol-water mixtures at 650 nm. The focused laser line intensity on the image captured by the camera module was fitted to a Lorentzian function to monitor peak intensity location over time. This, coupled with the selection of multiple test “channels” in the program and an adjustable sample area cutoff region helped to increase the signal to noise ratio and provide multiple datasets from individual samples. Data acquisition/fitting was taken at a rate of approximately one image per second across three channels in the image.

### B. Results and Discussion.

Here, the detection sensitivity of the system as a function of the capillary offset was investigated. The relative shift of the focused laser line position on the imaging chip between two analytes, water and 10% aqueous solution of ethylene glycol (EG), was used to determine the detection sensitivity. The experimentally obtained laser line positions on the imaging chip are shown in FIG. 4A-4F with a capillary tube offset at 0, 0.2, and 0.4 mm away from the principal axis of the probe laser beam. Here, the 10% EG sample with a RI greater than that of water creates a laser line at a lower position on the image plane for a nonzero offset of the capillary. The relative shift of the laser lines between water and 10% EG increases with increasing the capillary offset. As the height of the imaging chip was adjusted to accommodate the large change of the laser line positions when the capillary offset was changed from 0 to 0.4 mm, only the relative change in the laser line positions between water and 10% EG are comparable.

In addition, we conducted Zemax POP simulations to confirm the system design and predict measurement trends under the experimental conditions. The simulated laser line positions are shown in the images in FIG. 4G-4L with a capillary tube offset at 0, 0.2, and 0.4 mm for water and 10% EG, respectively. The simulated image also indicates a higher sensitivity, i.e., larger relative line shifts between water and 10% EG for a larger offset of the capillary from the laser principal axis, which is in agreement with the experimental results.

To obtain quantitative results on the shifts of the focused laser line, the position of the simulated laser line was determined by locating the position of the highest irradiance on the image plane. Tracking the shifts of the peak irradiance location across capillary tube offsets and sample RI changes was achieved by setting a defined aperture size on the imaging plane and measuring the displacement of the peak irradiance location between two samples with RIs of 1.3315 and 1.3408 to simulate water and 10% ethylene glycol at 650 nm. This method was used because Zemax defines the origin of the POP grid as the centroid of the beam, regardless of where the peak irradiance is located. In this case, both the change in sample RI and capillary tube offset cause the shift of the origin on the plot. Therefore, a fixed boundary of the image plane was required to provide relative shifts with a consistent reference.

Each value of the focused laser line location obtained from the water (RI=1.3315) configuration was compared to the value from the 10% EG (RI=1.3408). The relative shifts in the laser line positions were plotted against the capillary tube offset (FIG. 4M). For the seven capillary tube offset configurations tested (0, 0.05, 0.15, 0.2, 0.3, 0.4, and 0.5 mm), a linear trend (R²=0.9943) was observed in the simulations, showing that the relative laser line shifts increased linearly with the capillary tube offset. Similarly, the experimental results also demonstrated a linear relationship (R²=0.9387) between the relative laser line shift and the capillary tube offset. Therefore, higher sensitivity (i.e., a larger shift) can be achieved with greater capillary tube offsets; however, the variation in the measurements (i.e., the standard deviation) also increased due to the degradation of image quality as the offset increased. As seen in FIG. 4A-4F, the laser lines progressively spread out over a larger area on the imaging plane as the capillary offset increased. This trend observed in the experimental results was further confirmed by Zemax POP simulations (FIG. 4G-4L).

This phenomenon can be attributed to the fact that the imaging plane was set at only a single fixed location throughout the measurements. For the experiment, the imaging plane was set using water with 0 mm offset of the capillary. When either the RI or the offset changed, both the vertical position of the focused laser line and the focal length were affected, resulting in the spreading of the laser lines. As the position of the laser line is tracked by finding the peak of the intensity distribution, one can still measure the change in the RI precisely as long as the line intensity distribution can be well defined. An optimal capillary offset was found to be 0.4 mm, as offsets larger than this introduced excessive noise, making it difficult to obtain consistent data.

The small discrepancies between the simulation and experimental results, as shown in FIG. 4M, could be attributed to several factors, such as challenges in precisely locating the light source due to the housing of the semiconductor laser gain medium, potential inaccuracies in modeling the capillary tube, and the assumption of homogeneity of the samples in the simulation. Despite these limitations, the simulation provides a robust comparative framework, supporting a practical understanding of the system's behavior under different conditions.

Solutions of varying percentages of EG in water were used to create samples with different RIs. FIG. 4N shows a clear shift in the height of the focused laser line for solutions with 40%, 30%, 20%, 10% of EG in water measured under the condition where the capillary had an offset of 0.4 mm away from the principal axis of the laser beam. As the RIs of those samples at 650 nm are not available from literature, we measured them based on their relative position to water and acetonitrile, which have known RIs at 650 nm. The results shown in FIG. 4N demonstrated the capability of the system for monitoring RI changes in a wide range.

Using solutions of low (93 mg/dL) and high (632 mg/dL) fibrinogen control plasma, we evaluated and validated the capability of the system for measuring the coagulation of different plasma samples under controlled conditions. The coagulation process was initiated by exposing a 4 μL plasma sample to a thromboplastin-DS coated capillary tube through a capillary effect. The interior coating of each capillary tube was done under the same condition with 2 μL of a fixed thromboplastin-DS concentration (56 μg/mL), designed to trigger the clotting cascade consistently across each sample. The coagulation progresses of both types of fibrinogen plasma samples were monitored across three independent tests (n=3), conducted at a stable temperature of 21° C.

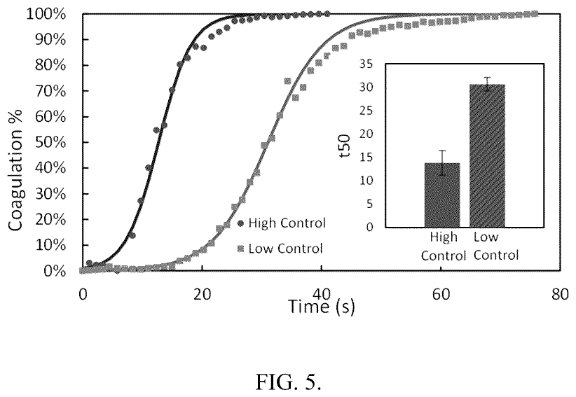

A sigmoidal function has been used to fit plasma coagulation curves because these curves naturally reflect the dynamic, time-dependent process of clot formation [22]. The coagulation process can be broken down into three phases. Initially, there's a lag phase where little activity is detected as thromboplastin-DS has just been introduced [23, 24]. This is followed by a rapid propagation phase as the coagulation cascade accelerates, characterized by rapid increase in prothrombin conversion and solid clot formation [25, 26]. Finally, the curve flattens out in a termination phase, where thrombin generation is halted, and any residual proteases are inhibited [27, 28]. The shifts in the probe laser line positions due to the coagulation-induced changes in the RI of the plasma samples were measured with our system. The time dependence of the shift clearly showed three coagulation phases (FIG. 5). This S-shaped progression curve is well-represented by a sigmoidal function:

$$C ( t ) = \frac{C}{1 + {e}^{- k ( t - {t}_{50} )}} \qquad Eq . 1$$

where C(t) represents the percentage of coagulation over time. The parameter C denotes the amplitude, which is reached when the laser line position stops shifting (presumably when coagulation reaches 100%). The steepness of the curve is governed by the parameter k, which determines how quickly the propagation phase occurs. Lastly, the time t₅₀ marks the inflection point, indicating the time at which the rate of coagulation is at its maximum.

To fit the experimental data with this function, the laser line position was tracked over time during coagulation, and nonlinear regression was applied to determine the best-fitting values for k and t₅₀. The value of C is predefined as 100%, representing the maximum level of coagulation. The initial flat section of the curve, or lag time, indicates how long it takes for the clotting process to initiate. The k-value reveals how rapidly the coagulation cascade progresses, while t₅₀ is particularly important because it shows the time at which clot formation is most active.

A distinction between the high and low fibrinogen controls shown in FIG. 5 was observed, with the time to reach 50% coagulation recorded at approximately 13.85 seconds for the high control and 30.61 seconds for the low control (FIG. 5). The nonlinear regression fit yielded k-values of approximately 0.3256/s±0.0837/s across all high fibrinogen controls and 0.1372/s±0.0569/s for the low fibrinogen controls, reflecting the difference in the rate of coagulation progression between the two samples. These findings confirm the expected differences in coagulation dynamics based on fibrinogen levels and highlight the applicability of the system developed in this study for coagulation time measurements.

Although the RI of whole blood can vary among individuals due to differences in overall composition (e.g., hemoglobin, oxygen saturation, and protein concentrations) [29], the approach described here is expected to accommodate these variations, as it relies on tracking relative (rather than absolute) changes in RI to determine coagulation time. Additionally, the current system design allows for optimization of the glass capillary offset to accommodate expected analyte RI differences in cases where the difference is too small or large. In this study, the transition from uncoagulated plasma to coagulated plasma was successfully monitored.

In summary, these experiments present a new mechanism for constructing a portable, compact device designed for precise measurement of the coagulation process. The mechanism uniquely leverages changes in RI to quantify coagulation time, setting it apart from existing approaches. The capillary tube used in this system is designed to serve multiple functions: (1) minimizing the required sample volume; (2) acting as a cylindrical lens to focus the probe beam onto an imaging chip, eliminating the need for additional optics. This configuration not only reduces system cost but also enhances accuracy by improving the precision of beam position determination compared to an unfocused beam; (3) increasing detection sensitivity by magnifying changes in probe beam position through an optimized offset of the capillary tube relative to the probe laser. The system has demonstrated robust performance in laboratory settings, showcasing its potential as a reliable tool for coagulation analysis. The work herein demonstrates a compact system based on a novel detection mechanism for coagulation measurements.

### REFERENCES

### Addin En.Reflist

1. Packebush, M., et al., Scientific Reports, 2023. 13.
2. Ware, K. M., et al., American Journal of Hypertension, 2015. 28 (2): p. 182-189.
3. Chaudry, R. S. M. U. H. M. B., N.C.f.B. Information, Editor. 2023, StatPearls: National Library of Medicine.
4. Hirsh, J. and J. F. Cade, M. Birnstingl, Editor. 1973, Butterworth-Heinemann. p. 46-74.
5. Skinner, S. C., et al., Kidney Medicine, 2021. 3 (4): p. 607-618.
6. Hansson, K. M., et al., Biosensors and Bioelectronics, 1999. 14 (8): p. 671-682.
7. Gage, B. F., S. D. Fihn, and R. H. White, The American Journal of Medicine, 2000. 109 (6): p. 481-488.
8. Bhutta, R. A., et al. Apr. 7, 2025]; Available from: URL labpedia.net/blood-coagulation-factors-and-their-interpretations/.
9. Zaidi, S. R., Preeti, Interpretation of Blood Clotting Studies and Values (PT, PTT, aPTT, INR, Anti-Factor Xa, D-Dimer), in StatPearls [Internet]. 2024, StatPearls Publishing.
10. Tripodi, A., et al., Alimentary Pharmacology & Therapeutics, 2007. 26 (2): p. 141-148.
11. Tripathi, M. M., et al., Sci Rep, 2017. 7 (1): p. 9169.
12. Mayo Clinic, S., Tests & Procedures, 2024.
13. Yang, R., M. Zubair, and L. Moosavi, Prothrombin time, in StatPearls [Internet]. 2024, StatPearls Publishing.
14. Donkin, R., Y. L. Fung, and I. Singh, Fibrinogen, Coagulation, and Ageing, in Biochemistry and Cell Biology of Ageing: Part III Biomedical Science, J. R. Harris and V. I. Korolchuk, Editors. 2023, Springer International Publishing: Cham. p. 313-342.
15. St John, A., National Library of Medicine, 2010 (0159-8090 (Print)).
16. Saha, A., et al., Evolving Paradigm of Prothrombin Time Diagnostics with Its Growing Clinical Relevance towards Cardio-Compromised and COVID-19 Affected Population. LID-10.3390/s21082636 [doi] LID-2636. National Library of Medicine, 2021 (1424-8220 (Electronic)).
17. Nizamov, S., S. D. Sazdovska, and V. M. Mirsky, Analytica Chimica Acta, 2022. 1204: p. 339633.
18. Jones, A. R., Progress in Energy and Combustion Science, 1999. 25 (1): p. 1-53.
19. Nyholm, L., Analyst, 2005. 130 (5): p. 599-605.
20. Grady, M., et al., National Library of Medicine, 2014 (1932-2968 (Electronic)).
21. Hoffman, M. S. F., et al., Expert Review of Medical Devices, 2023. 20 (1): p. 5-16.
22. Trine, P. V., et al., Journal of Biomedical Optics, 2000. 5 (1): p. 51-55.
23. Butenas, S., K. G. Mann, and Butenas, Biochemistry (Moscow), 2002. 67 (1): p. 3-12.
24. Butenas, S., C. van 't Veer, and K. G. Mann, Journal of Biological Chemistry, 1997. 272 (34): p. 21527-21533.
25. Lawson, J. H., et al., Journal of Biological Chemistry, 1994. 269 (37): p. 23357-23366.
26. Rand, M. D., et al., Blood clotting in minimally altered whole blood. 1996.
27. Olson, S. T., I. Björk, and J. D. Shore, Academic Press, 1993, p. 525-559.
28. Girard, T. J., et al., Nature, 1989. 338 (6215): p. 518-520.
29. Zhernovaya, O., et al., Physics in medicine and biology, 2011. 56: p. 4013-21.
30. Elblbesy, M. A., AIMS Biophysics, 2021. 8 (1): p. 57-65.
31. Hammer, M., et al., Applied Optics, 1998. 37: p. 7410-7418.

1. A blood coagulation monitoring device comprising:
a divergent probe beam source producing a divergent probe beam when in use;
a sample holder, the sample holder configured to position a capillary containing a sample offset from the center of the divergent probe beam;
a sensor configured to detect a portion of the divergent probe beam;
the probe beam source and capillary are positioned such that a portion of the divergent probe beam passes through the capillary containing the sample and converges onto the sensor.

2. The device of claim 1, wherein the divergent probe beam source is a diode laser.

3. The device of claim 1, wherein the sensor is an imaging chip.

4. The device of claim 1, further comprising a control unit operably connected to the sensor.

5. A blood collection capillary comprising a collection segment coated with calcium chelator and a detection segment coated with a coagulation activator, wherein the capillary is configured to receive a sample in the collection segment and during operation transfer the sample to the detection segment.

6. The capillary of claim 5, wherein the calcium chelator is citrate or oxalate.

7. The capillary of claim 5, wherein the coagulation activator is tissue factor, CaCl₂, calcium gluconate, silica, celite, kaolin, or ellagic acid.

8. The capillary of claim 5, wherein the inner diameter of the capillary is 0.2 to 3 mm.

9. The capillary of claim 5, wherein the volume of the detecting segment is 1.0 to 10 μL.

10. A method for monitoring blood coagulation comprising:
(i) exposing a capillary containing a blood sample to a diverging probe beam that is configured to pass through the blood sample, the capillary converging the diverging probe beam, and the converged probe beam impinging on a sensor configured to detect changes in refraction of a portion of the diverging probe beam;
(ii) detecting changes in refractive index of the sample by detecting a change in refraction angle of the probe beam as determined by analyzing data collected by the sensor;
(iii) assessing blood coagulation processes in the sample based on the change in refraction angle of the diverging probe beam as it passes through the sample to determine the change in refractive index of the blood sample.

11. The method of claim 10, wherein the sensor is an imaging chip.

12. The method of claim 10, wherein the sensor collects data continuously.

13. The method of claim 10, wherein the sample volume is 1 to 10 μL.

14. The method of claim 10, wherein the refractive index of the blood sample is used to indicate disease presence.

15. The method of claim 10, wherein the refractive index of the blood sample is used to monitor disease treatment progression.

16. The method of claim 14, wherein diseases include cancer, anemia, hypoproteinemia, polycythemia vera, hyperproteinemia, hyperlipidemia, and liver disease.
